(19) United States
(12) Patent Application Publication
(10) Pub. No.: US 2025/0290163 A1
(43) Pub. Date: Sep. 18, 2025

(54) MEASUREMENT DEVICE, MEASUREMENT METHOD, AND PROGRAM

(71) Applicant: Yokogawa Electric Corporation, Tokyo (JP)

(72) Inventors: Keita SODA, Tokyo (JP); Takahisa KONISHI, Tokyo (JP); Yasuhiro MATSUI, Tokyo (JP)

(21) Appl. No.: 19/070,163

(22) Filed: Mar. 4, 2025

(30) Foreign Application Priority Data
Mar. 12, 2024 (JP) 2024-038433

(51) Int. Cl. C12Q 1/70; B01L 7/00; C12N 15/10; C12Q 1/6806; C12Q 1/6851; G16B 30/00

(52) U.S. Cl. CPC C12Q 1/70; B01L 7/52; C12N 15/1017; C12N 15/1096; C12Q 1/6806; C12Q 1/6851; G16B 30/00; B01L 2200/028; B01L 2300/0681; B01L 2300/18

(57) ABSTRACT

A measurement device according to an embodiment includes a virus disruption device and a PCR device. The virus disruption device elutes a nucleic acid of a virus contained in water by heating water to be measured to a specified temperature in less than a specified time. The PCR device measures an amount of virus contained in water by performing one-step RT-qPCR with respect to the water in which the nucleic acid is eluted by the virus disruption device.

### CROSS-REFERENCE TO RELATED APPLICATION(S)

The present application claims priority to and incorporates by reference the entire contents of Japanese Patent Application No. 2024-038433 filed in Japan on Mar. 12, 2024.

### FIELD

The present invention relates to a measurement device, a measurement method, and a program.

### BACKGROUND

Conventionally, water treatment systems that purify treated water using a membrane filtration device equipped with a filtration membrane have been known. In water treatment systems, a chemical solution such as sodium hypochlorite, is injected into the membrane filtration device to remove organic matter contained in the treated water (for example, JP-A-2020-104093).

Moreover, a method using columns have been known as a method of extracting and purifying nucleic acids (for example, JP-A-2002-360245).

Furthermore, as a method of detecting a trace amount of RNA molecular specimen, the one-step reverse transcription polymerase chain reaction (RT-PCR) and two-step RT-PCR have been known (for example, JP-A-2023-8970). Moreover, in JP-T-2012-525837, one-step RT-quantitative PCR (qPCR) is described.

To efficiently measure viruses in a water treatment system, the use of RT-qPCR is considered. In such cases, a process of eluting or extracting nucleic acids from supply water or filtered water of the membrane filtration device becomes necessary. The method extracting and purifying nucleic acids using a column described in JP-A-2002-360245 has a problem that it takes a long time.

In one aspect, an object is to perform virus detection in a water treatment system in a shorter time.

### SUMMARY

According to an aspect of an embodiment, a measurement device includes: a virus disruption device that elutes, by heating water to be measured to a specified temperature in less than a specified time, a nucleic acid of a virus contained in the water; and a PCR device that performs a one-step RT-qPCR with respect to the water in which the nucleic acid is eluted by the virus disruption device to measure an amount of the virus contained in the water.

According to an aspect of an embodiment, a measurement method that is performed by a measurement device, includes: heating water to be measured to a specified temperature in less than a specified time to elute a nucleic acid of a virus contained in the water; and measuring an amount of the virus contained in the water by performing one-step RT-qPCR with respect to the water in which the nucleic acid is eluted.

According to an aspect of an embodiment, a non-transitory computer readable recording medium having stored therein a program that causes a computer to perform a process including: calculating an index to evaluate a performance of a membrane filtration device based on a first amount that is an amount of virus in membrane-filtration supply water supplied to a membrane filtration device measured by a PCR device measuring an amount of virus contained in water by performing one-step RT-qPCR with respect to the water in which a nucleic acid of a virus is eluted by heating the water to be measured to a specified temperature in less than a specified time, and a second amount that is an amount of virus in membrane permeate water subjected to membrane filtration by the membrane filtration device measured by the PCR device.

### BRIEF DESCRIPTION OF THE DRAWINGS

FIG. 1 is a diagram illustrating a relationship between injection of sodium hypochlorite and residual chlorine;

FIG. 2 is a diagram illustrating a configuration example of a water treatment system according to an embodiment;

FIG. 3 is a diagram illustrating a configuration example of a ultrafiltration (UF) membrane filtration system according to the embodiment;

FIG. 4 is a diagram illustrating a configuration example of a reverse osmosis (RO) membrane filtration system according to the embodiment;

FIG. 5 is a detailed configuration example of the RO-membrane filtration device according to the embodiment;

FIG. 6 is a block diagram illustrating a configuration example of a control device according to the embodiment;

FIG. 7 is a block diagram illustrating a configuration example of a measurement device according to the embodiment;

FIG. 8 is a diagram illustrating a relationship between heating temperature in high temperature processing (HTP) and pepper mild mottle virus (PMMoV);

FIG. 9 is a diagram illustrating a relationship between heating time in HTP and PMMoV;

FIG. 10 is a block diagram illustrating a configuration example of an evaluation device according to the embodiment;

FIG. 11 is a flowchart illustrating a flow of processing of the measurement device according to the embodiment;

FIG. 12 is a flowchart illustrating an example of flow of processing to determine an injection amount of a chemical according to the embodiment;

FIG. 13 is a flowchart illustrating an example of flow of processing to determine a cleaning method according to the embodiment; and

FIG. 14 is a diagram explaining a hardware configuration example of a control device.

### DETAILED DESCRIPTION OF THE PREFERRED EMBODIMENTS

Hereinafter, embodiments of a measurement device, a measurement method, and a program disclosed in the present application will be explained in detail with reference to the drawings. The embodiments explained herein are not intended to limit the present invention. Moreover, identical reference symbols are assigned to identical components, and duplicated explanation will be omitted appropriately. Moreover, the respective embodiments may be combined within arrange not causing a contradiction.

### 1. Introduction

Water treatment of sewage, drainage of rainwater, and the like is generally broadly categorized into three main processes including primary treatment, secondary treatment, and tertiary treatment.

In the primary treatment, removal of large solid materials such as foreign objects contained in wastewater is performed. In the secondary treatment, organic matter that could not be eliminated in the primary treatment is removed by using microorganisms (bacteria). In the secondary treatment, for example, activated sludge treatment, nitrification-denitrification treatment, and the like are performed. In the tertiary treatment, removal of suspended solids that could not be eliminated in the secondary treatment is performed by sediment removal. In the tertiary treatment, removal of suspended solids by sand filtration or membrane filtration is performed.

In the secondary treatment, a large number of microorganisms are present in the wastewater, and these microorganisms oxidize and decompose organic matter. Subsequently, the microorganisms are precipitated and removed as sludge. The waste water after the removal of the sludge contains residual microorganisms that have not been fully precipitated, viruses that adhere to bacteria, and the like.

By injecting chlorine (sodium hypochlorite) into this treated wastewater (secondary treated water or tertiary treated water), microorganisms, viruses, and the like are removed. This enables the water treatment system 1 to prevent the proliferation of microorganisms and viruses and to suppress clogging of the filtration membranes in subsequent membrane filtration processing.

At this point, an excessive amount of sodium hypochlorite that exceeds a stable threshold is injected into the wastewater. By injecting such an excessive amount of sodium hypochlorite, microorganisms and viruses are more reliably removed.

When sodium hypochlorite (chlorine) is added to the wastewater, combined chlorine (chloramine) is generated from ammonia (NH⁴—N).

Generally, the types of combined chlorine present in water vary depending on the following reaction Equations 1 to 4 and the chemical equilibrium.

NH₃+HOCl→NH₂Cl+H₂O (1)

NH₂Cl+HOCl→NHCl₂+H₂O (2)

NHCl₂+HOCl→NCl₃+H₂O (3)

2NHCl₂+OH⁻→N₂+HOCl+2H⁺+3Cl⁻ (4)

As shown in reaction equations 1 to 4, ammonia (NH₃) is primarily converted into three types of combined chlorine-monochloramine (NH₂Cl), dichloramine (NHCl₂), and trichloramine (NCl₃) depending on water quality conditions.

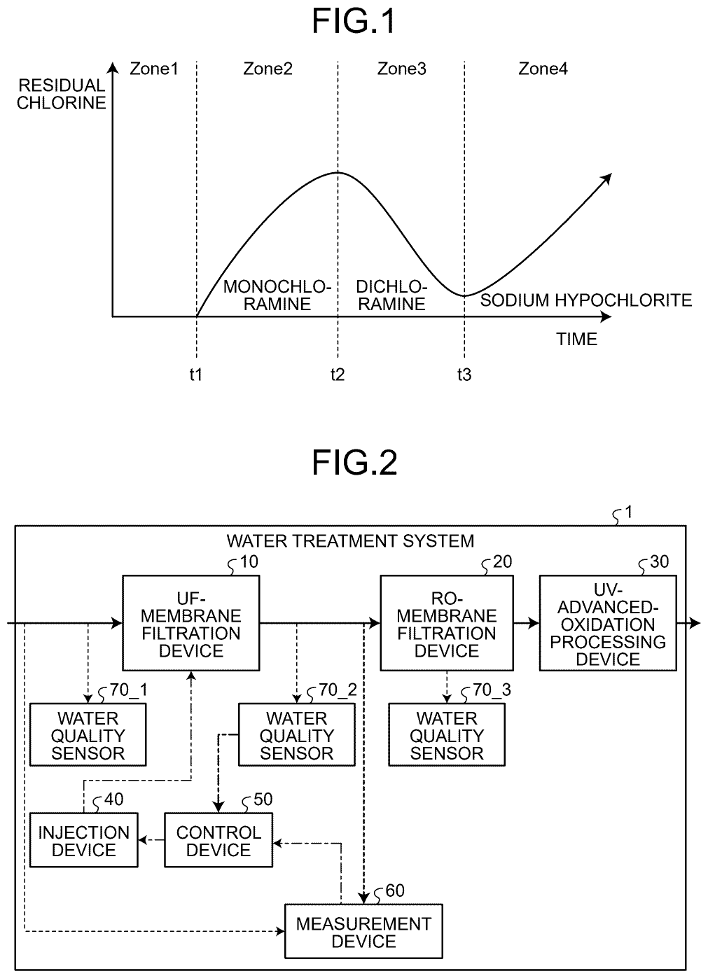

FIG. 1 is a diagram illustrating a relationship between injection of sodium hypochlorite and residual chlorine. In this example, the graph shows residual chlorine when a constant amount of sodium hypochlorite is injected into ammonia water per unit of time. In the graph in FIG. 1, the horizontal axis represents time, and the vertical axis represents residual chlorine.

Specifically, in a period from the start of sodium hypochlorite injection until a first time t1 (Zone 1), because sodium hypochlorite is consumed by microorganisms, sodium hypochlorite is not detected in the treated water. The first time t1 varies depending on an amount of microorganisms contained in the treated water.

In a period thereafter (after the first time t1) until a second time t2 (Zone 2), the reaction described in equation 1 becomes dominant, and ammonia reacts with sodium hypochlorite to form monochloramine, which begins to be detected.

As sodium hypochlorite continues to be further injected, the reaction described in equation 2 becomes dominant during in a period from the second time t2 to a third time t3 (Zone 3), and dichloramine begins to be detected. Thereafter, as sodium hypochlorite continues to be further injected, sodium hypochlorite itself begins to be detected after a time t3. The second time t2 to the third time t3 varies depending on an amount of ammonia contained in the wastewater or the like.

The time t2 cannot be clearly defined, and the timing of dichloramine formation varies depending on changes in water quality. Therefore, to suppress dichloramine formation, water quality is managed by an experienced administrator.

The amounts of microorganisms and ammonia contained in the water to be treated vary depending on the quality of the wastewater and the treatment and the secondary treatment. To remove microorganisms more reliably, an excess amount of sodium hypochlorite and ammonia (for example, ammonium sulfate or ammonium chloride) is typically injected into the water to be treated.

As described, as an excessive amount of sodium hypochlorite or ammonium sulfate (or ammonium chloride) is injected, the consumption of these substances includes, resulting in higher costs.

Furthermore, the excessive injection of sodium hypochlorite leads to an increase of N-nitrosodimethylamine (NDMA). NDMA is formed when chloramine reacts with NDMA precursors. It is known that dichloramine particularly causes an explosive increase of NDMA production.

For example, in the United States, a standard has been set for NDMA to be 10 ng/L or less in drinking water. NDMA generated by the injection of sodium hypochlorite cannot be removed through membrane filtration. Instead, NDMA is decomposed through ultraviolet (UV) advanced oxidation (post-AOP, photolysis, advanced oxidation) process in a subsequent stage to the membrane filtration process.

When an amount of sodium hypochlorite injected into the water to be treated is large, it not only increases the cost due to an increase in consumption of sodium hypochlorite but also increases a load on UV advanced oxidation necessary for NDMA removal. If the load on UV advanced oxidation increases, processing time and costs for the water treatment system increase.

On the other hand, when an amount of sodium hypochlorite injected into the wastewater is insufficient, it causes a problem that microorganisms in the wastewater cannot be removed completely. Microorganisms that are not completely removed can reduce the permeability of the filtration membranes used in the membrane filtration process, and the quality of the treated water (recycled water) can be degraded.

As described, in the water treatment system, there is a trade-off between the quality of recycled water and the cost of recycling wastewater. Therefore, it is desired to control the respective treatment processes to maintain the quality of recycled water while minimizing costs.

Moreover, an excessive injection amount causes a problem that the filtration membrane (for example, a microfiltration (MF) membrane, a UF membrane, a nanofiltration (NF) membrane, or an RO membrane) is more prone to damage because more chlorine remains in the wastewater. To prevent this problem, as a material for filtration membranes, a chlorine-resistant, durable material (for example, polyvinylidene fluoride (PVDF)) is becoming mainstream. NF membranes and RO membranes are predominantly made of cross-linked aromatic polyamide, which is known to be subject to damage such as oxidative degradation caused by dichloramine.

Even with a membrane made of PVDF, if the membrane surface or pores are clogged, the filtered water volume decreases. To maintain the filtered water volume, it is necessary to supply wastewater to the filtration membrane at a higher pressure. That is, it is necessary for a pump supplying wastewater to the filtration membrane to operate at a higher pressure, and the energy consumption of the pump increases.

As described, use of PVDF does not necessarily contribute to cost reduction of the water treatment system, and more appropriate improvement measures are expected.

Filtration membranes (for example, MF membranes or UF membranes) are regularly cleaned to resolve clogging. Examples of cleaning of MF or UF membranes include backwashing (cleaning using filtered water), chemical cleaning (cleaning using chemicals, such as sulfuric acid, citric acid, and hypochlorite) (maintenance cleaning (MC)), chemical cleaning applying long-term immersion in a high concentration chemical (recovery cleaning (RC)), and the like.

In the membrane filtration processing, the operating sequence including filtration using a filtration membrane (for example, MF membrane or UF membrane), the cleaning described above, and other processes is set (initially configured) during a design stage. Generally, this initial configuration (settings based on initial conditions) is rarely changed or optimized by a user.

Because an NF membrane or an RO membrane arranged downstream of an MF membrane or a UF membrane is more susceptible to damage from the higher concentration of chloramine or dichloramine, it is necessary to control the chloramine concentration within an appropriate concentration range to prevent damage to the NF membrane or the RO membrane.

Therefore, in the present embodiment, a control device that controls water treatment acquires water quality information regarding supply water (corresponding to the wastewater described above) (including information about viruses) at an inlet portion of a membrane filtration device. The control device determines an amount of sodium hypochlorite to be injected to the membrane filtration device according to the water quality information.

Thus, the control device can suppress chloramine that is generated by the injection of sodium hypochlorite to a predetermined concentration (for example, Zone 1 in which monochloramine is formed), thereby preventing formation of NDMA.

Moreover, because the control device determines an amount of sodium hypochlorite to be injected depending on the water quality, an excessive amount of sodium hypochlorite is not to be injected to the membrane filtration device. Thus, the control device can control a usage of sodium hypochlorite more appropriately, resulting in a further reduction in costs.

Particularly, in the present embodiment, an amount of viruses can be measured. This enables to evaluate virus removal performance of the membrane filtration device. Furthermore, by referring to the virus removal performance of the membrane filtration device, the control device can control the usage of sodium hypochlorite more preferably.

### 2. Configuration of Embodiment

A configuration of the water treatment system will be explained using FIG. 2. FIG. 2 is a diagram illustrating a configuration example of the water treatment system according to the embodiment.

The water treatment system 1 illustrated in FIG. 2 includes a UF-membrane filtration device 10, an RO-membrane filtration device 20, a UV-advanced-oxidation processing device 30, an injection device 40, a control device 50, a measurement device 60, a water quality sensor 70_1, a water quality sensor 70_2, and a water quality sensor 70_3. The water treatment system 1 regenerates treated wastewater, such as sewage and drainage of rainwater, into domestic water or drinking water.

To the water treatment system 1, treated water obtained by subjecting wastewater to the activated sludge treatment, the nitrification-denitrification reaction treatment, and the like (that is, treated water subjected to the primary treatment and the secondary treatment) is supplied. The water treatment system 1 performs membrane treatment and the like to the supplied treated water. That is, the water treatment system 1 performs water treatment for the purpose of reusing mainly the secondary treated water and the tertiary treated water described above.

The treated water processed by the water treatment system 1 is disinfected, for example, with chlorine or the like, and is then used as domestic water or drinking water.

The activated sludge treatment, the nitrification-denitrification reaction treatment, and the like are performed, for example, at a wastewater treatment facility. The water treatment system 1 performs the membrane treatment and the like, for example, on wastewater treated at the wastewater treatment facility.

The UF-membrane filtration device 10 removes microorganisms and particulate matter from the supplied water through a filtration membrane. The filtration membrane of the UF-membrane filtration device 10 is, for example, ultrafiltration membrane (UF membrane). In the following, for simplicity of explanation, it is supposed that the water treatment system 1 performs membrane filtration using the UF membrane, but the water treatment system 1 may perform membrane filtration using a filtration membrane other than the UF membrane. For example, the water treatment system 1 can perform membrane filtration using microfiltration membrane (MF membrane) and the like. When the water treatment system 1 performs membrane filtration using a membrane other than the MF membrane, the UF membrane in the explanation below is replaced with the MF membrane.

To the RO-membrane filtration device 20, water treated by the UF-membrane filtration device 10 is supplied. The RO-membrane filtration device 20 removes impurities, such as ions and salts, from the supply water. The RO-membrane filtration device 20 includes an RO membrane. In the following, for simplicity of explanation, it is supposed that the water treatment system 1 performs membrane filtration using the RO membrane, but the water treatment system 1 may perform membrane filtration by using a filtration membrane other than the RO membrane. For example, the water treatment system 1 can perform membrane filtration using the NF membrane. When the water treatment system 1 performs membrane filtration using the NF membrane, the RO membrane in the following explanation will be replaced with the NF membrane.

To the UV-advanced-oxidation processing device 30, water treated by the RO-membrane filtration device 20 is supplied. The UV-advanced-oxidation processing device 30 performs advanced oxidization treatment by irradiating UV to the supplied water, to remove NDMA and the like.

Supply water that is supplied to the UF-membrane filtration device 10 is also denoted as membrane-filtration supply water. Supply water that is supplied to the RO-membrane filtration device 20 is denoted as reverse-osmosis-membrane supply water. Supply water that is supplied to the UV-advanced-oxidation processing device 30 is also denoted as UV supply water.

The water quality sensor 70_1 is arranged at an inlet portion of the UF-membrane filtration device 10. The water quality sensor 70_1 measures the water quality of the membrane-filtration supply water.

The water quality sensor 70_1 measures at least one of a water temperature, a pH value, an oxidation-Reduction potential (ORP), an ammonia nitrogen content, a nitrogen compound content, turbidity, ultraviolet absorbance, electrical conductivity, and a total organic carbon (TOC) value of the membrane-filtration supply water. The water quality sensor 70_1 outputs the measurement result to the control device 50 as water quality information.

The measurement device 60 measures an amount of viruses in membrane-filtration supply water supplied to the UF-membrane filtration device 10 and membrane-filtered permeate water subjected to membrane filtration by the UF-membrane filtration device 10. Moreover, the measurement device 60 evaluates a filtration performance of the UF-membrane filtration device 10 based on a measurement result. The measurement device 60 outputs the measurement result and the evaluation result to the control device 50 as the water quality information.

The injection device 40 injects a chemical solution to the inlet portion of the UF-membrane filtration device 10 in accordance with an instruction from the control device 50. The injection device 40 injects, for example, sodium hypochlorite to the inlet portion of the UF-membrane filtration device 10. Moreover, the injection device 40 injects, for example, a chemical solution, such as ammonium sulfate or ammonium chloride, into the inlet portion of the UF-membrane filtration device 10 other than sodium hypochlorite.

The control device 50 controls the respective components of the water treatment system 1. The control device 50 according to the present embodiment performs control processing illustrated in FIG. 2.

The control device 50 first acquires water quality information of membrane-filtration supply water from the water quality sensor 70_1 and the measurement device 60, as the control processing.

The control device 50 determines an injection amount of sodium hypochlorite according to the water quality information. For example, the control device 50 determines the injection amount of sodium hypochlorite such that more microorganisms contained in the membrane-filtration supply water are removed while minimizing the formation of dichloramine. More specifically, the control device 50 determines the injection amount such that a state of the membrane-filtration supply water to which sodium hypochlorite is injected becomes a state in zone 1 described above.

The control device 50 instructs the injection device 40 to inject the determined injection amount into the inlet portion of the UF-membrane filtration device 10.

Thus, the water treatment system 1 can achieve removal of microorganisms and the suppression of NDMA formation while suppressing the injection amount of sodium hypochlorite, and can thereby further reduce the treatment costs.

A solid arrow (for example, an arrow entering the RO-membrane filtration device 20 from the UF-membrane filtration device 10) in FIG. 2 represents a flow of treated water subject to treatment such as filtration. Moreover, a dotted arrow for example, an arrow entering the measurement device 60 and the respective water quality sensors) indicates a flow of treated water obtained for measurement purposes. A dash-dotted arrow (for example, an arrow entering the control device 50) represents a flow of signals and data. Furthermore, a dash-dot-dot arrow (for example, an arrow entering the UF-membrane filtration device 10 from the injection device 40) signifies the injection of a chemical solution.

The respective devices included in the water treatment system 1 in FIG. 2 will be explained in detail.

### UF-Membrane Filtration Device 10

The UF-membrane filtration device 10 includes a UF membrane (not illustrated). The UF-membrane filtration device 10 performs membrane filtration using the UF membrane with respect to the UF-membrane supply water, to remove microorganisms and particulate matter. The UF-membrane filtration device 10 supplies membrane-filtered permeate water after membrane filtration (UF-membrane permeate water) to the RO-membrane filtration device 20.

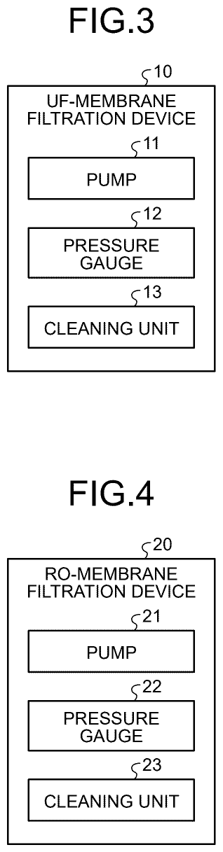

FIG. 3 is a diagram illustrating a configuration example of the UF-membrane filtration device according to the embodiment. As illustrated in FIG. 3, the UF-membrane filtration device 10 includes, for example, a pump 11, a pressure gauge 12, and a cleaning unit 13.

The pump 11 is a feeder pump that supplies UF-membrane supply water to the UF-membrane.

The pressure gauge 12 measures a pressure at the inlet portion and a pressure at an outlet portion of the UF-membrane filtration device 10. For example, the pressure gauge 12 measures a pressure of the UF-membrane supply water to the UF-membrane filtration device 10. The pressure gauge 12 measures a pressure of the UF-membrane permeate water that has passed through the UF-membrane filtration device 10. The pressure gauge 12 outputs the respective measured pressures to the control device 50.

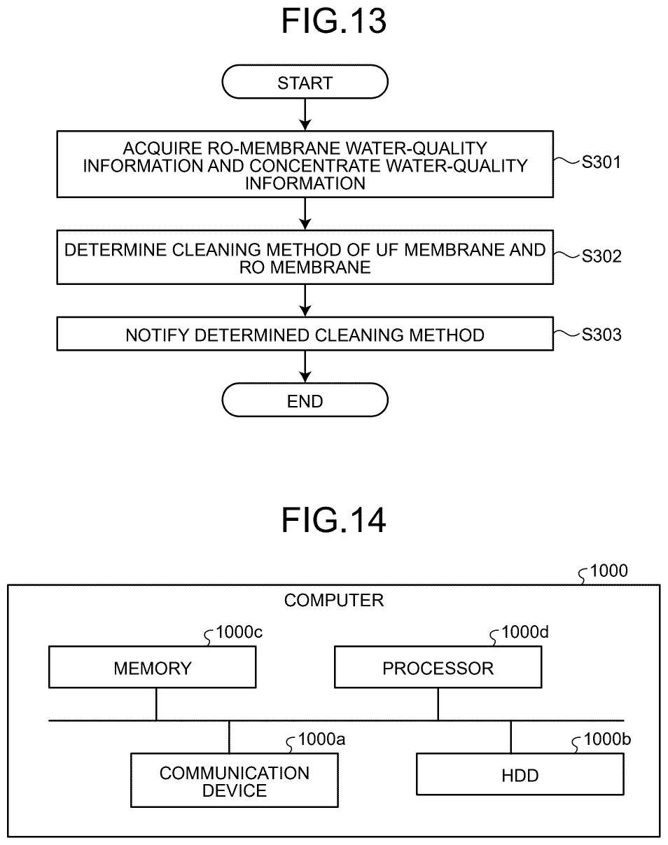

The cleaning unit 13 illustrated in FIG. 13 performs cleaning of the UF membrane in accordance with an instruction from the control device 50. The cleaning unit 13 performs, for example, backwashing, MC, RC, or the like.

### RO-Membrane Filtration Device 20

It is a diagram illustrating a configuration example of the RO-membrane filtration device according to the embodiment. The RO-membrane filtration device 20 illustrated in FIG. 4 includes an RO membrane (not illustrated). The RO-membrane filtration device 20 performs desalination (removal of ionic substance) from the RO-membrane supply water using the RO membrane.

The RO-membrane filtration device 20 includes, for example, a pump 21, a pressure gauge 22, and a cleaning unit 23.

The pump 21 is a feeder pump to supply the RO supply water to the RO membrane. The pump 21 may include multiple feeder pumps.

The pressure gauge 22 measures a pressure at the inlet portion and a pressure at an outlet portion of the RO-membrane filtration device 20. For example, the pressure gauge 22 measures a pressure of the RO-membrane supply water to the RO-membrane filtration device 20. The pressure gauge 22 measures a pressure of the RO-membrane permeate water that has passed through the RO-membrane filtration device 20. The pressure gauge 22 outputs the respective measured pressures to the control device 50.

The cleaning unit 23 illustrated in FIG. 4 performs cleaning of the RO membrane in accordance with an instruction from the control device 50. The cleaning unit 23 performs, for example, MC, RC, and the like.

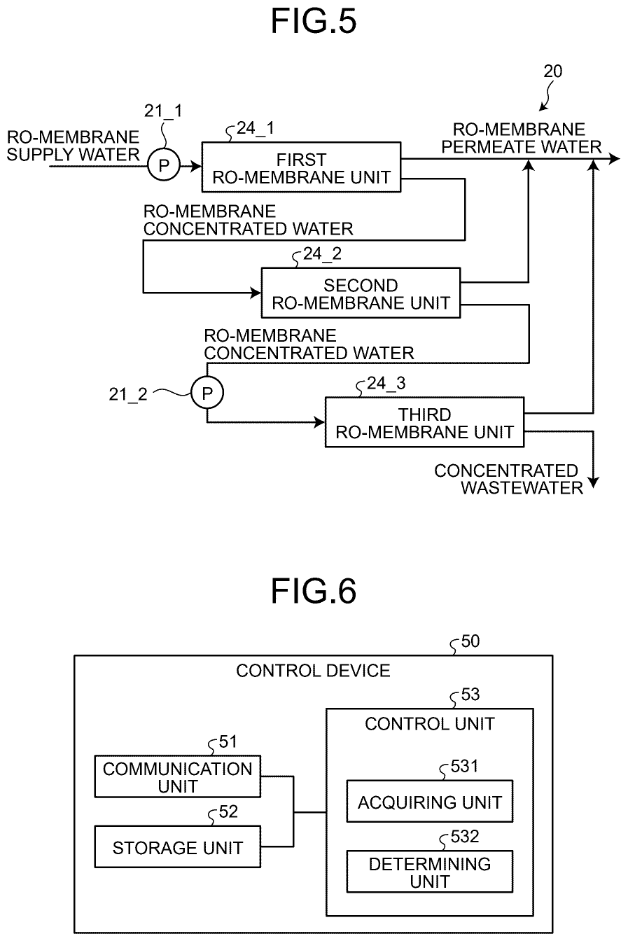

FIG. 5 is a diagram illustrating a detailed configuration example of the RO-membrane filtration device according to the embodiment. The RO-membrane filtration device 20 illustrated in FIG. 5 includes first to third RO membrane units 24_1 to 24_3.

The first RO-membrane unit 24_1 includes an RO membrane (not illustrated). To the first RO-membrane unit 24_1, the RO-membrane supply water is supplied using, for example, a feeder pump 21_1 (one example of the pump 21). The first RO-membrane unit 24_1 separates the RO supply water into RO permeate water (RO-membrane filtered water) and RO-membrane concentrated water. The first RO-membrane unit 24_1 supplies the RO concentrated water to the second RO-membrane unit 24_2.

The second RO-membrane unit 24_2 includes an RO membrane (not illustrated). To the second RO-membrane unit 24_2, RO-membrane concentrated water is supplied from the first RO-membrane unit 24_1. The second RO-membrane unit 24_2 separates the RO-membrane concentrated water into RO-membrane permeate water and RO-membrane concentrated water. The second RO-membrane unit 24_2 supplies the RO-membrane concentrated water to the third RO-membrane unit 24_3.;

The third RO-membrane unit 24_3 includes an RO membrane (not illustrated). To the third RO-membrane unit 24_3, the RO-membrane concentrated water is supplied from the second RO-membrane unit 24_2 using, for example, a feeder pump 21_2 (one example of the pump 21). The third RO-membrane unit 24_3 separates the RO-membrane concentrated water into RO-membrane permeate water and concentrated wastewater using the RO membrane. The third RO-membrane unit 24_3 drains the concentrated wastewater to the outside of the water treatment system 1.

The RO-membrane filtration device 20 supplies the RO membrane permeate water to the UV-advanced-oxidation processing device 30.

### UV-Advanced-Oxidation Processing Device 30

The UV-advanced-oxidation processing device 30 in FIG. 5 performs UV-advance oxidation process (AOP) (advanced oxidation processing using ultraviolet rays) with respect to the UV supply water. Thus, the UV-advanced-oxidation processing device 30 oxidizes and decomposes trace chemical substances (for example, NDMA) contained in the UV supply water.

### Water Quality Sensor 70 (Water Quality Sensors 70_1 to 70_3)

The water quality sensor 70 measures a water quality of supply water, filtered water (permeate water), or the like of respective parts of the water treatment system 1. In the example of FIG. 2, the water treatment system 1 includes water quality sensors 70_1 to 70_3.

The water quality sensor 70_1 measures a water quality at the inlet portion of the UF-membrane filtration device 10. The water quality sensor 70_1 measures a water quality of the UF-membrane supply water. The water quality sensor 70_1 measures, for example, at least one of water temperature, pH, ORP, ammonia nitrogen and the like, nitrogen compound, turbidity, ultraviolet absorbance, electrical conductivity, and TOC of the UF-membrane supply water. The water quality sensor 70_1 may include multiple sensors (for example, a thermometer, a pH meter, a turbidimeter, a conductivity meter, and the like) to measure these.

The water quality sensor 70_2 measures a water quality at an inlet portion of RO-membrane filtration device 20 (or an outlet portion of the UF-membrane filtration device 10). The water quality sensor 70_2 measures a water quality of the RO-membrane supply water (or the UF-membrane permeate water). The water quality sensor 70_2 measures at least one of water temperature, pH, ORP, TOC, and microorganism data (at least one of virus, bacteria, and adenosine triphosphate (ATP)) of the RO-membrane supply water. The water quality sensor 70_2 may include multiple sensors (for example, a thermometer, a pH meter, and the like) to measure these.

The water quality sensor 70_3 measures a water quality of the RO-membrane concentrated water (or the concentrated wastewater) of the RO-membrane filtration device 20. The water quality sensor 70_3 measures at least one of water temperature, pH, ORP, TOC, and microorganism data (at least one of virus, bacteria, and adenosine triphosphate (ATP)) of the RO-membrane concentrated water. The water quality sensor 70_3 may include multiple sensors (for example, a thermometer, a pH meter, and the like) to measure these.

The water quality sensor 70 included in the water treatment system 1 is not limited to the example in FIG. 2. For example, the water treatment system 1 may include the water quality sensor 70 that is not illustrated in FIG. 2, such as the water quality sensor 70 that measures a water quality of the UV supply water.

### Injection Device 40

The injection device 40 injects various kinds of chemical solutions at the inlet portion of the UF-membrane filtration device 10. The injection device 40 injects a chemical solution in accordance with an instruction of the control device 50. For example, the injection device 40 injects sodium hypochlorite into the membrane-filtration supply water. Moreover, the injection device 40 injects ammonium sulfate or ammonium chloride into the membrane-filtration supply water.

The injection device 40 included in the water treatment system 1 is not limited to the example in FIG. 2. For example, the water treatment system 1 may include the injection device 40 not illustrated in FIG. 2, such as the injection device 40 that injects a chemical solution into the UV supply water.

As described, the injection device 40 injects ammonium sulfate (or ammonium chloride) and sodium hypochlorite at the inlet portion of the UF-membrane filtration device 10 (in other words, the UF membrane) to form chloramine.

Chloramine is a combined chlorine formed by reacting chlorine (sodium hypochlorite exists as hypochlorous acid HOCl or hypochlorite ion OCl⁻ in water (the membrane-filtration supply water) with ammonia.

Generally, types of combined chlorine present in water differ depending on reaction equations 1 to 4 described above and chemical equilibria.

As shown in reaction equations 1 to 4, ammonia (NH₃) is converted into three main types of combined chlorine (monochloramine (NH₂Cl), dichloramine (NHCl₂), and trichloramine (NCl₃)) based on water quality conditions.

The oxidative power and the disinfectant effect of chloramine increase in the order of monochloramine, dichloramine, and trichloramine. That is, dichloramine has greater oxidative power and disinfectant effect than monochloramine, and trichloramine has greater power and effect than dichloramine. Moreover, hypochlorous acid and hypochlorite ion exhibit the highest disinfectant effect.

When the amount of chlorine (sodium hypochlorite) injected into the membrane-filtration supply water increases, and a chlorine to ammonia ratio rises, ammonia converts to monochloramine, then progresses to dichloramine and trichloramine, to be removed from the water as nitrogen gas, liberated in as hypochlorous acid or hypochlorite ion.

In the present embodiment, the chlorine to ammonia ratio (Cl₂:NH₃) in the membrane-filtration supply water is set to 1:2.5 to 1:3. That is, the control device 50 sets NH₃/Cl₂ in the membrane-filtration supply water to 2.5 to 3, and sets the injection amount of ammonium sulfate (or ammonium chloride) and sodium hypochlorite for the purpose of generating monochloramine.

The oxidative power of dichloramine, trichloramine, and free chlorine is stronger than that of monochloramine and, as reported in Reference [0095], it is known to cause degradation of RO membranes made of an aromatic polyamide-based material.

Furthermore, as reported in Reference [0097], conversion to NDMA is known to be accelerated by reactions with nitrogen compounds (for example, N₂, NH₃, NO₂, NO₃, N₂O, and the like) in the presence of dichloramine.

When NDMA formation is accelerated, the UV-advanced-oxidation processing device 30 needs to increase a UV lamp irradiation energy in the UV-AOP to promote NDMA decomposition, a processing cost for this process increases.

On the other hand, wastewater subject to treatment of the water treatment system 1 contains a high concentration of nitrogen compound. Moreover, components of and the nitrogen concentration of the nitrogen compound in wastewater vary.

In conventional water treatment systems, the analysis and management of nitrogen compounds have not been performed in real time, and have not been reflected in treatment of wastewater. In practice, the conventional water treatment systems have been injecting an excessive amount of sodium hypochlorite into the wastewater (membrane-filtration supply water). As described, the conventional water treatment systems tend to aim for chloramine formation with a margin to accommodate fluctuations in the water quality of the wastewater.

This is because, if the water quality of the wastewater fluctuates to a state with excess nitrogen or significant chlorine consumption, an insufficient chlorine injection amount can cause a failure to form chloramines that suppress biological activity because chlorine is consumed by nitrogen. As described, if the chlorine injection amount is insufficient for a wastewater quality, a desired biological disinfection effect is difficult to achieve, and clogging of a filtration membrane in a subsequent state (for example, UF membrane) can progress, making it difficult to continue operation of the UF-membrane filtration device 10.

Therefore, the conventional water treatment system have been injecting excessive sodium hypochlorite to achieve more reliable chloramine formation not affected by the water quality fluctuations of wastewater.

As described, the conventional water treatment system has become a chloramine-dependent system with excessive chlorine, and can lead to an increase in treatment costs from following three perspectives.
1. Increased chemical cost necessary for chloramine generation
2. Increased power consumption for UV-AOP oxidation of NDMA and the like resulting from reactions with dichloramine and the like
3. Degradation of an RO membrane due to decomposition of aromatic polyamides (that is, increase in RO membrane replacement cost

The water treatment system 1 according to the present embodiment aims to move away from chlorine-dependent membrane filtration operation caused by excessive injection of a chlorine agent (sodium hypochlorite) by appropriately controlling the injection amount of sodium hypochlorite according to the water quality of the wastewater (membrane-filtration supply water).

### REFERENCES

The Impact of Monochloramines and Dichloramines on Reverse Osmosis Membranes in Wastewater Potable Reuse Process Trains: Pilot-scale Study (Environ. Sci. Water Res. Technol., 2020, 6, pp. 1336-1346) Updated Reaction Pathway for Dichloramine Decomposition: Formation of Reactive Nitrogen Species and N-Nitrosodimethylamine (Environ. Sci. Technol., 2021, 55, pp. 1740-1749)

As described above, the control device 50 according to the present embodiment is configured to maintain the concentration of monochloramine generated in wastewater (membrane-filtration supply water) to transition the water treatment system 1 from a chloramine-dependent system to a low-chloramine-dependent system.

More specifically, the control device 50 acquires a water quality of the membrane-filtration supply water, and determines an injection amount of sodium hypochlorite such that the concentration of monochloramine to be generated falls within a predetermined concentration range (for example, zone 1 described above) depending on the water quality. Moreover, the control device 50 determines an injection amount of ammonium sulfate or ammonium chloride similarly.

For example, the control device 50 predicts a nitrogen compound in the membrane-filtration supply water based on the water quality of the membrane-filtration supply water, and determines an injection rate of chemicals (sodium hypochlorite and ammonium sulfate (or ammonium chloride)) to form monochloramine at a predetermined concentration. The prediction of a nitrogen compound may be performed using a simulation, or may be performed using machine learning (AI model).

Thus, the water treatment system 1 can avoid excessive injection of sodium hypochlorite and ammonium sulfate (or ammonium chloride), and can control the injection amount of these chemicals appropriately, thereby reducing the injection amount of the chemicals.

Furthermore, the water treatment system 1 can keep the dichloramine concentration low by maintaining the monochloramine concentration within a predetermined concentration range. Thus, the water treatment system 1 can suppress generation of NDMA and the like, and can suppress an increase of power consumption for UV-AOP. Moreover, the water treatment system 1 can suppress deterioration of an RO membrane caused by decomposition of aromatic polyamide of the RO membrane, thereby reducing the replacement cost of the RO membrane.

Depending on the monochloramine concentration in the membrane-filtration supply water, residual microorganisms can increase the likelihood of UF membrane clogging.

Accordingly, the control device 50 according to the present embodiment predicts a clogging state (permeability) of the UF membrane of the UF-membrane filtration device 10. The control device 50 changes a cleaning frequency and a cleaning method (concentration of a chemical to be used, or the like of the UF-membrane filtration device 10 based on the prediction result. The control device 50 changes an amount of a chemical (sodium hypochlorite and ammonium sulfate (ammonium chloride)) to be injected into the membrane-filtration supply water according to the prediction result.

### Control Device 50

Hereinafter, an example of a configuration of the control device 50 that performs these will be explained.

FIG. 6 is a block diagram illustrating a configuration example of the control device 50 according to the present embodiment. The control device 50 illustrated in FIG. 6 includes a communication unit 51, a storage unit 52, and a control unit 53.

The communication unit 51 performs data communication with other devices. For example, the communication unit 51 performs communications with the respective devices of the water treatment system 1.

The storage unit 52 stores various kinds of information to be referred to when the control unit 53 operates, and various kinds of information acquired when the control unit 53 operates. The storage unit 52 can be implemented by a semiconductor memory device, such as a random access memory (RAM) and a flash memory, a storage device, such as a hard disk and an optical disk, or the like. In the example in FIG. 6, the storage unit 52 is arranged inside the control device 50, but it may be arranged outside the control device 50, or multiple storage units may be arranged.

The control unit 53 controls the entire control device 50 and the water treatment system 1. The control unit 53 includes an acquiring unit 531 and a determining unit 532. The control unit 53 can be implemented by an electrical circuit, such as a central processing unit (CPU) and a micro processing unit (MPU), or an integrated circuit, such as an application specific integrated circuit (ASIC) and a field programmable gate array (FPGA).

The acquiring unit 531 acquires various kinds of information from the water treatment system 1. The acquiring unit 531 acquires, for example, pressure information regarding respective pressures measured by the pressure gauge 22. The acquiring unit 531 acquires, for example, UF-membrane pressure information regarding a pressure of the UF-membrane supply water. The acquiring unit 531 acquires, for example, RO-membrane pressure information regarding a pressure of the RO-membrane supply water.

The acquiring unit 531 acquires the water quality information regarding the water quality from, for example, the measurement device 60 and the water quality sensors 70_1 to 70_3. The acquiring unit 531 acquires UF-membrane water-quality information regarding a water quality of the UF-membrane supply water, for example, from the water sensor 70_1. The acquiring unit 531 acquires RO-membrane water-quality information regarding a water quality of the RO-membrane supply water, for example, from the water sensor 70_2. The acquiring unit 531 acquires concentrate water-quality information regarding a water quality of the RO-membrane concentrated water, for example, from the water sensor 70_3. The acquiring unit 531 acquires a measurement result of viruses or an evaluation result of performance of the UF-membrane filtration device from the measurement device 60.

The acquiring unit 531 outputs the respective acquired information to the determining unit 532.

The determining unit 532 determines an injection amount of a chemical (sodium hypochlorite and ammonium sulfate (or ammonium chloride)) based on the information acquired by the acquiring unit 531. For example, the determining unit 532 determines the injection amount of a chemical using machine learning model that outputs an injection amount based on the input water quality information and the like.

The determining unit 532 determines a cleaning method of at least one of the UF membrane of the UF-membrane filtration device 10 and the RO membrane of the RO-membrane filtration device 20 based on at least one of the RO-membrane water-quality information and the concentrate water-quality information. Alternatively, the determining unit 532 may determine a cleaning method of at least one of the UF membrane and the RO membrane based on the pressure information. For example, the determining unit 532 determines a cleaning method using a machine learning model that outputs a cleaning method based on the input water quality information and the like.

The determining unit 532 determines at least one of frequency (timing) of implementation of MC and RC, and the concentration of a chemical used for cleaning, as the cleaning method for the UF membrane and the RO membrane.

### Measurement Device 60

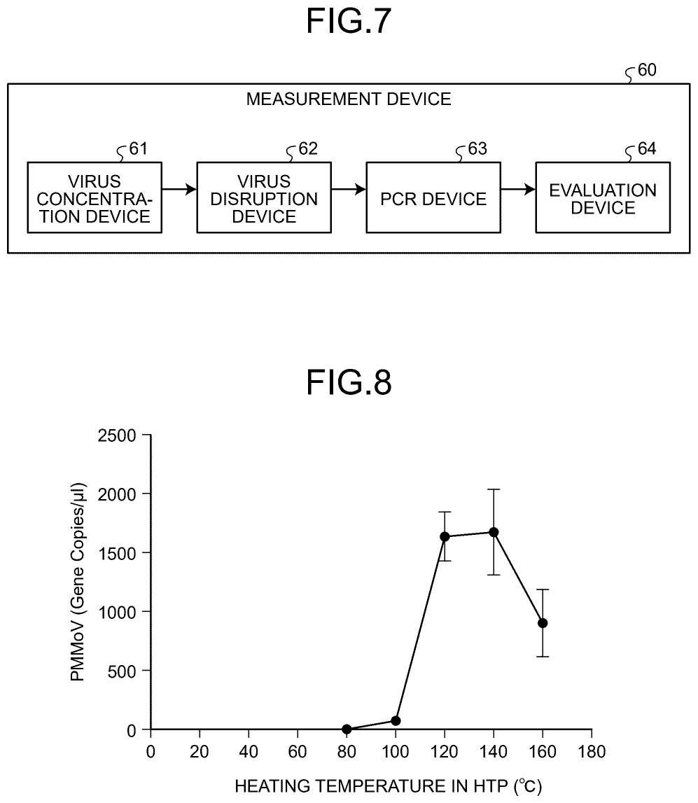

A configuration of the measurement device 60 will be explained using FIG. 7. FIG. 7 is a block diagram illustrating a configuration example of the measurement device according to the embodiment.

As illustrated in FIG. 7, the measurement device 60 includes a virus concentration device 61, a virus disruption device 62, a PCR device 63, and an evaluation device 64. First, water to be measured is injected into the virus concentration device 61. For example, to the virus concentration device 61, the membrane-filtration supply water supplied to the UF-membrane filtration device 10, and the membrane permeate supply water subjected to the membrane filtration by the UF-membrane filtration device 10 are injected.

The virus concentration device 61 concentrates viruses in the injected water. For example, the virus concentration device 61 may be a concentrating pipette device manufactured by InnovaPrep (registered trademark) (reference [0129]).

### REFERENCES

CONCENTRATING PIPETTE SELECT, INNOVAPREP (URL:https://www.innovaprep.com/products/concentrating-pipette)

The virus disruption device 62 performs processing of disrupting viruses and eluting nucleic acids with respect to the water in which the viruses are concentrated by the virus concentration device 61. For example, the virus disruption device 62 elutes nucleic acids by the method described in Reference [0133], that is, high temperature pressure method (HTP).

It is presumed that treating water containing viruses under a high-temperature and high-pressure condition in HTP dissolves the viruses, and it is expected that the nucleic acids contained within the viruses will be eluted into the water at the same time. Thus, elution of nucleic acids derived from viruses from the viruses can be achieved.

### Reference

JP-A-2012-157265

Reference describes the elution of nucleic acids from fungi and bacteria using HTP. In this embodiment, HTP is applied to the virus disruption device 62 with a protocol (parameter settings) suitable for the elution of nucleic acids from viruses, enabling the elution of nucleic acids from the viruses.

That is, the virus disruption device 62 introduces water to be measured into a container, tightly seals the container, and heats the water in the container in a sealed state to a specified temperature above 100° C., in less than the specified time.

The specified maximum temperature and the specified time are set as parameters of HTP. For example, the specified temperature is within a range between 120° C. and 160° C., and the specified time is within a range between 5 seconds and 30 seconds. Particularly, the specified time may be 140° C., and the specified time may be 15 seconds.

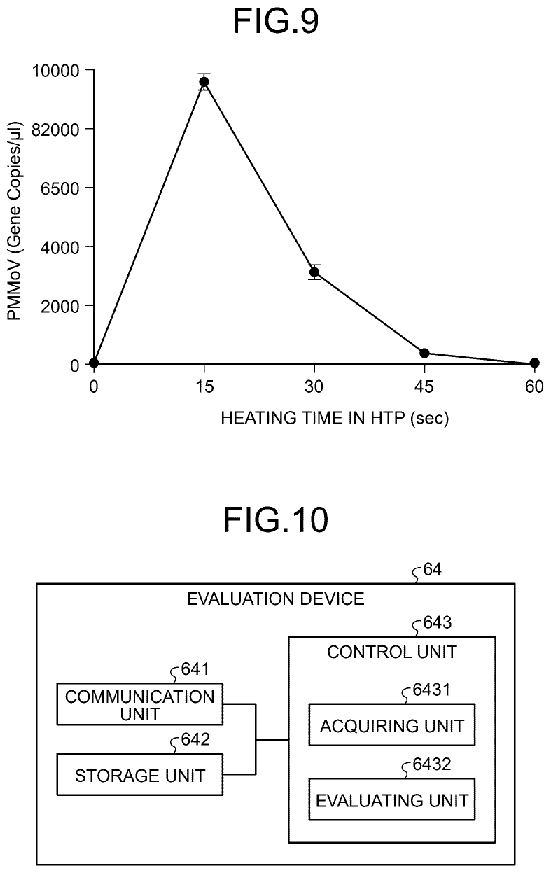

The specified maximum temperature and the specified time may be determined based on a result obtained by measuring pepper mild mottle virus (PMMoV) when the heating temperature and the heating time of HTP are changed. Examples of the measurement results are illustrated in FIG. 8 and FIG. 9. For example, the heating temperature and the heating time that maximize the amount of detected PMMoV may be set to the specified maximum temperature and specified time.

FIG. 8 is a diagram illustrating a relationship between the heating temperature of HTP and PMMoV. As illustrated in FIG. 8, the amount of detected PMMoV becomes sufficiently large when the heating temperature is within a range of 120° C. and 160° C. FIG. 9 is a diagram illustrating a relationship between the heating time in HTP and PMMoV. As illustrated in FIG. 9, the amount of detected PMMoV becomes sufficiently large when the heating time is within a range between 5 seconds and 30 seconds (particularly, around 15 seconds).

The PCR device 63 performs a one-step RT-qPCR on the nucleic acids eluted by the virus disruption device 62 and measures the amount of virus.

The two-step RT-qPCR involves two steps including CDNA synthesis (reverse transcription) and a quantitative test by qPCR. Therefore, the advantage of two-step RT-qPCR is that the cDNA can be stored for a long time, making it useful for screening. On the other hand, the disadvantage of two-step RT-qPCR is that it takes a long time.

In contrast, the one-step RT-qPCR has an advantage that time necessary therefor is short compared to the two-step RT-qPCR.

In the present embodiment, because the nucleic acids have been eluted by the virus disruption device 62, time associated with column work used in an RNA extraction kit and the like in the two-step RT-qPCR becomes unnecessary. As a result, the measurement of viruses in the water treatment system 1 can be performed in a short time.

For example, the one-step RT-qPCR is achieved by using an HTP dedicated tube, and processing water containing viruses by HTP. First, the PCR device 63 has a tube containing a reaction solution (primer, probe, and RT-qPCR enzyme) that includes transcriptase necessary for reverse transcription and polymerase necessary for qPCR, in a liquid or lyophilized form equipped in advance.

The PCR device 63 aliquots a sample (water containing eluted nucleic acids, that is, water containing nucleic acids derived from viruses) received from the virus disruption device 62 to a reaction solution in a liquid or lyophilized form in the tube, and mixes the solution uniformly, and then performs qPCR with the real time PCR device.

Moreover, the PCR device 63 can perform absolute quantification, equipped with multiple tubes containing the reaction solution, by adding each of multiple virus concentration solutions for which the concentration of virus is adjusted, for example, to match the PMMoV standard. In each of the tubes, the PMMoV standard is, for example, one of nine variations below.
1.0×108 gene copies/μ1
1.0×107 gene copies/μ1
1.0×106 gene copies/μ1
1.0×10⁵ gene copies/μ1
1.0×10⁴ gene copies/μ1
1.0×103 gene copies/μ1
1.0×102 gene copies/μ1
1.0×101 gene copies/μ1
1.0×100 gene copies/μ1

The evaluation device 64 evaluates the virus removal performance of the UF-membrane filtration device 10 based on the measurement result by the PCR device 63.

FIG. 10 is a block diagram illustrating a configuration example of the evaluation device 64 according to the present embodiment. The evaluation device 64 illustrated in FIG. 10 includes a communication unit 641, a storage unit 642, and a control unit 643.

The communication unit 641 performs data communication with other devices. For example, the communication unit 641 performs communication with the PCR device 63 and the control device 50.

The storage unit 642 stores various kinds of information that is referred to when the control unit 643 operates, and various kinds of information acquired when the control unit 643 operates. The storage unit 642 can be implemented by a semiconductor memory device, such as a RAM and a flash memory, a storage device, such as a hard disk and an optical disk, or the like. In the example in FIG. 7, the storage unit 52 is arranged inside the evaluation device 64, but it may be arranged outside the evaluation device 64, or multiple storage units may be arranged.

The control unit 643 controls the entire evaluation device 64. The control unit 643 includes an acquiring unit 6431 and an evaluating unit 6432. The control unit 643 can be implemented by an electrical circuit, such as a CPU and an MPU, or an integrated circuit, such as an ASIC and an FPGA.

The acquiring unit 6431 acquires a measurement result from the PCR device 63.

The evaluating unit 6432 evaluates the virus removal performance of the UF-membrane filtration device 10 based on the measurement result acquired by the acquiring unit 6431.

For example, the acquiring unit 6431 acquires a first amount that is an amount of virus in the membrane-filtration supply water supplied to the UF-membrane filtration device 10 measured by the PCR device 63, and a second virus amount that is an amount of virus in the membrane permeate water subjected to membrane filtration by the UF-membrane filtration device 10 measured by the PCR device 63.

The evaluating unit 6432 more highly evaluates the performance of the UF-membrane filtration device 10 as a degree of deviation between the first amount and the second amount increases (where the first amount>the second amount). For example, the evaluating unit 6432 calculates a value obtained by subtracting the second amount from the first amount as an index indicating the performance of the UF-membrane filtration device 10. Moreover, for example, the evaluating unit 6432 calculates a value obtained by dividing the first amount by the second amount as an index indicating the performance of the UF-membrane filtration device 10.

The measurement device 60 includes a casing. In the casing, the virus concentration device 61, the virus disruption device 62, the PCR device 63, and the evaluation device 64 are housed. That is, the measurement device 60 is a package of the respective devices. Thus, the measurement device 60 becomes portable. However, the measurement device 60 can be assembled. That is, the respective devices are attachable to the casing and the detachable from the casing.

The packaged measurement device 60 is attachable to the water treatment system 1, and detachable from the water treatment system 1. The measurement device 60 is only required to include an interface to connect with the control device 50 in a data communication enabled manner, and an inlet to take in water.

For example, in cases of disasters or the like, there is a risk of deterioration in sanitary conditions. To prevent this, it is essential to quantitatively evaluate an extent of virus presence in water at the disaster site. Because the measurement device 60 of the present embodiment packages the respective devices in an attachable and detachable manner, the portability is improved, and assessment of water at a disaster site is possible.

### 3. Flow of Processing of Embodiment

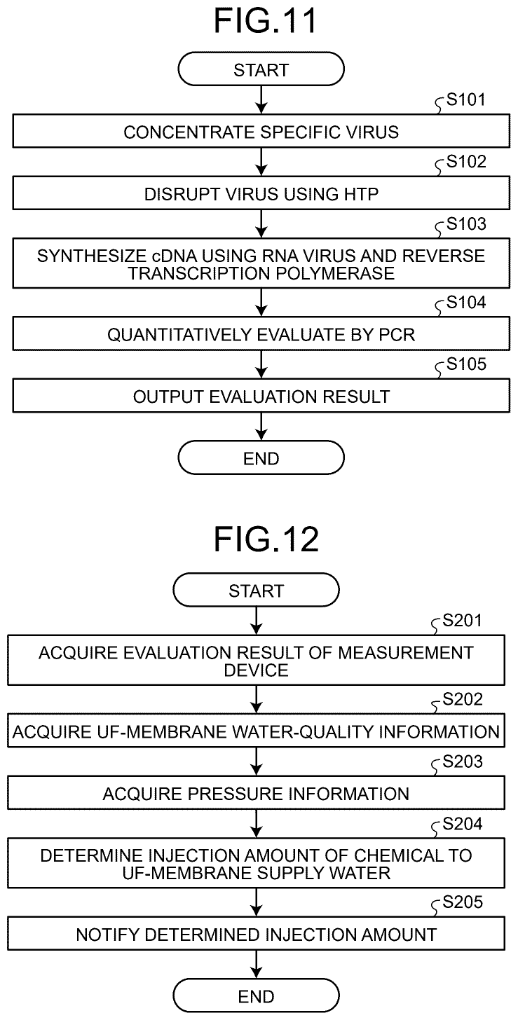

FIG. 11 is a flowchart illustrating an example of a flow of processing of the measurement device according to the embodiment. As illustrated in FIG. 11, the measurement device 60 performs processing of concentrating a specific virus with respect to injected water (step S101).

The measurement device 60 performs disruption of viruses using HTP with respect to the water in which the virus is concentrated (step S102). Thus, nucleic acids of the virus are eluted.

The measurement device 60 subjects an RNA virus contained in the water in which the nucleic acids of the virus are eluted to the reverse transcription (cDNA synthesis) using the reverse transcriptase polymerase (step S103).

The measurement device 60 performs quantitative evaluation on cDNA by PCR (step S104). The measurement device 60 outputs the evaluation result (step S105). The measurement device 60 can output the evaluation result to the control device 50.

The measurement device 60 can intermittently repeat the processing illustrated in FIG. 11 while the water treatment system 1 is in operation. The measurement device 60 can calculate the performance of the UF-membrane filtration device 10 in real time by performing the evaluation of the membrane-filtration supply water supplied to the UF-membrane filtration device 10 and the membrane permeate water subjected to the membrane filtration by the UF-membrane filtration device 10 intermittently.

FIG. 12 is a flowchart illustrating an example of a flow of processing of determining an injection amount of a chemical according to the embodiment. The processing in FIG. 12 is repeatedly perform by the control device 50 while the water treatment is being performed by the water treatment system 1.

As illustrated in FIG. 12, the control device 50 acquires the evaluation result of the measurement device 60 (step S201). Moreover, the control device 50 acquires the UF-membrane water-quality information regarding the water quality of the UF-membrane supply water from the water quality sensor 70_1 (step S202). Moreover, the control device 50 acquires the pressure information from the pressure gauge 12 of the UF-membrane filtration device 10 and the pressure gauge 22 of the RO-membrane filtration device 20 (step S203).

The control device 50 determines the injection amount of the chemical to the UF-membrane supply water using, for example, a machine learning model based on the acquired information (step S204). The control device 50 notifies the determined injection amount to the injection device 40 (step S205).

It is a flowchart illustrating an example of a flow of processing of determining a cleaning method according to the embodiment. The processing in FIG. 13 is repeatedly performed by the control device 50, for example, while the water treatment is being performed by the water treatment system 1.

As illustrated in FIG. 13, the control device 50 acquires the RO-membrane water-quality information regarding the water quality of the RO-membrane supply water and the concentration water-quality information regarding the water quality of the RO-membrane concentrated water from the water quality sensors 70_2 and 70_3 (step S301).

The control device 50 determines the cleaning method (for example, cleaning timing and a concentration of a chemical used for cleaning, and the like), for example, using a machine learning model based on the RO-membrane water-quality information and the concentrate water-quality information (step S302).

The control device 50 notifies the determined cleaning method to the cleaning unit 13 of the UF-membrane filtration device 10 and the cleaning unit 23 of the RO-membrane filtration device 20 (step S303). The cleaning unit 13 and the cleaning unit 23 perform cleaning of the UF membrane and the RO membrane according to the determined cleaning method.

### 4. System

Unless otherwise specified, the processing procedures, the control procedures, the specific names, and the information including various kinds of data and parameters illustrated in the above document and in the drawings may be arbitrarily changed.

Moreover, the respective components of the respective devices illustrated are of functional concept, and it is not necessarily required to be configured physically as illustrated. That is, specific forms of distribution and integration of the respective devices are not limited to the ones illustrated. That is, all or some thereof can be configured to be distributed or integrated functionally or physically in arbitrary units according to various kinds of loads, usage conditions, and the like.

Furthermore, as for the respective processing functions performed by the respective devices, all or an arbitrary part thereof can be implemented by a CPU and a program that is analyzed and executed by the CPU, or can be implemented as hardware by wired logic.

### 5. Hardware

Next, a hardware configuration example of the control device 50, which is an information providing device, and the evaluation device 64 will be explained. FIG. 14 is a diagram explaining a hardware configuration example of the control device 50 and the evaluation device 64. The control device 50 and the evaluation device 64 are implemented by a computer 1000 illustrated in FIG. 14.

As illustrated in FIG. 14, the computer 1000 includes a communication device 1000a, a hard disk drive (HDD) 1000b, a memory 1000c, and a processor 1000d. Moreover, the respective components illustrated in FIG. 14 are connected to one another through a bus or the like.

The communication device 1000a is a network interface card or the like, and performs communication with other servers. The HDD 1000b stores a program to operate the functions explained so far and a DB.

The processor 1000d reads the program that implements processing similar to that of the respective processing units from the HDD 1000b or the like and loads it to the memory 1000c, to thereby operate processes to implement the respective functions explained in FIG. 6, FIG. 10, and the like. For example, this process performs functions similar to those of the respective processing units of the control unit 53 and the control unit 643. Specifically, the processor 1000d reads out a program having functions similar to those of the respective processing units from the HDD 1000b or the like. The processor 1000d performs the process to perform the processing similar to that of the respective processing units.

As described, the computer 1000 operates as a device that performs various kinds of processing methods by reading and executing a program. Moreover, the computer 1000 can implement functions similar to the embodiment described above by reading the program described above from a recording medium by a medium reader device, and by executing the read program described above also. Other programs in the embodiments are not limited to be executed by the computer 1000. For example, the present invention can be applied similarly also to a case in which the program is executed by other computers or servers, or a case in which the program is executed by these in cooperation.

This program can be distributed through a network such as the Internet. Furthermore, this program can be recorded on a computer-readable recording medium, such as a hard disk, a flexible disk (FD), a compact disk read-only memory (CD-ROM), a magneto optical disk (MO), and a digital versatile disk (DVD), and can be executed by being read by a computer from the recording medium.

### 6. Others

Some examples of combinations of the disclosed technical features are described in the following.

According to an embodiment, it is possible to perform measurement of virus in a water treatment system in short time.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A measurement device comprising:
a virus disruption device that elutes, by heating water to be measured to a specified temperature in less than a specified time, a nucleic acid of a virus contained in the water; and
a PCR device that performs a one-step RT-qPCR with respect to the water in which the nucleic acid is eluted by the virus disruption device to measure an amount of the virus contained in the water.

2. The measurement device according to claim 1, wherein
the PCR device has a tube that contains a reaction solution for reverse transcription and qPCR, and adds the reaction solution in the tube to the water in which the nucleic acid is eluted by the virus disruption device.

3. The measurement device according to claim 1, wherein
the virus disruption device elutes the nucleic acid of the virus included in the water by heating the water to the specified temperature within a range between 120° C. and 160° C., in less than the specified time within a range between 5 seconds and 30 seconds.

4. The measurement device according to claim 2, wherein
the PCR device has the tube that contains the reaction solution in a lyophilized form.

5. The measurement device according to claim 1, further comprising
a virus concentration device that concentrates a virus in water acquired by a water treatment system, wherein
the virus disruption device performs processing of eluting a nucleic acid of the virus contained in the water in which the virus is concentrated by the virus concentration device.

6. The measurement device according to claim 1, further comprising
an evaluation device that calculates, based on a first amount that is an amount of virus in membrane-filtration supply water supplied to a membrane-filtration device measured by the PCR device and a second amount that is an amount of virus in membrane permeate water subjected to membrane filtration by the membrane filtration device measured by the PCR device, an index to evaluate a performance of the membrane filtration device.

7. The measurement device according to claim 1, further comprising
a casing that houses the virus disruption device and the PCR device.

8. The measurement device according to claim 7, wherein
the virus disruption device and the PCR device are attachable to the casing and detachable from the casing.

9. A measurement method that is performed by a measurement device, comprising:
heating water to be measured to a specified temperature in less than a specified time to elute a nucleic acid of a virus contained in the water; and
measuring an amount of the virus contained in the water by performing one-step RT-qPCR with respect to the water in which the nucleic acid is eluted.

10. A non-transitory computer readable recording medium having stored therein a program that causes a computer to perform a process including: calculating an index to evaluate a performance of a membrane filtration device based on a first amount that is an amount of virus in membrane-filtration supply water supplied to a membrane filtration device measured by a PCR device measuring an amount of virus contained in water by performing one-step RT-qPCR with respect to the water in which a nucleic acid of a virus is eluted by heating the water to be measured to a specified temperature in less than a specified time, and a second amount that is an amount of virus in membrane permeate water subjected to membrane filtration by the membrane filtration device measured by the PCR device.
